(19) United States
(12) Patent Application Publication
(10) Pub. No.: US 2007/0054393 A1
(43) Pub. Date: Mar. 8, 2007

(54) Dispensing device

(76) Inventors: Markus Kehlenbeck, Weyhe (DE); Dennis Pump, Hamburg (DE); Dagmar Weber, Hamburg (DE); Irmgard Lorenzen, Rabenholz (DE); Dagmar Lambrue, Fassberg (DE)

Correspondence Address: GRIFFIN & SZIPL, PC, SUITE PH-1, 2300 NINTH STREET, SOUTH, ARLINGTON, VA, 22204, US

(21) Appl. No.: 10/467,596

(22) PCT Filed: Feb. 14, 2002
(86) PCT No.: PCT/EP02/01544

§ 371 Date: Feb. 2, 2005

(30) Foreign Application Priority Data
Feb. 14, 2001 (DE) 101 06 713.5

(51) Int. Cl. C12M 1/26; B01L 3/02

(52) U.S. Cl. 435/309.1; 422/100

(57) ABSTRACT

The invention relates to a dispensing device for dispensing particle suspensions (12), especially cell suspensions, comprising a reservoir (10) for receiving the particle suspension (12). A suspending agent (26) is provided in the reservoir (10) that influences the particle suspension (12). The suspending agent (26) serves to preserve homogenous particle distribution in the particle suspension (12). A microdispenser (22) is connected to the reservoir (10) by a fluid element (18). The microdispenser serves for delivering microdroplets on a titer plate.

The invention relates to a dispensing device for the dispensing of particle suspensions, particularly of cell suspensions.

Particularly when conducting research in the field of active substances, a large number of active substances is tested nowadays by means of high-throughput screening. In doing so, a large number of samples is tested in a short time in an automated process. In high-throughput screening, the active substances are inserted into miniaturized microtitration plates or other suitable sample carriers with wells provided therein. Since the costs for the active substances, the storage of these substances in substance libraries and the disposal of the active substances after the test process are extremely expensive, the volumes of the individual wells are as small as possible. In modern titration plates, the volumes of the individual wells are often in the microliter and nanoliter range.

Thus, it is required that minimum quantities be inserted into the wells by pipetting or dispensing. A suitable pipetting device for active substances without sensitive particles is described e.g. in WO 96/24040.

Since the examination of particle suspensions, particularly of cell suspensions, has gained increased importance, it has become desirable to develop a device adapted to supply even minimum quantities of particle suspension into wells. In this regard, a particular problem resides in that e.g. cells and other particles which have low mechanical stability may be easily damaged when introduced into the wells. Especially, pipetting of liquid samples cannot be performed if the samples are particle suspensions since the particles have to pass the very narrow pipette opening both during the suctional intake into the pipette and during ejection from the pipette. In this regard, for instance, a considerable danger exists that the cells are affected by shearing forces and that the surface function of the cells is impaired. Thus, the pipetting device described in WO 96/24040 is not useful for particle suspensions.

Further, a system for the dispensing of liquids is known from WO 98/06821. In this system, one individual particle or one individual cell is dispensed per dispensing process. This known device is extremely complex and expensive. Such a device is particularly ill-suited for high-throughput screening since the filling of the individual wells which shall take up a specific number of particles or cells, is too time-consuming to allow e.g. 10,000 tests to be carried out per day in effective systems.

It is an object of the invention to provide a dispensing device for the dispensation of particle suspensions, particularly of cell suspensions, which is adapted to dispense minimum quantities over periods of several hours.

According to the invention, the above object is achieved by a dispensing device having the features mentioned in claim 1.

The dispensing device according to the invention comprises a reservoir for receiving a particle suspension, particularly a cell suspension. The particle suspension can further comprise synthetic microparticles such as beads, particularly polymeric beads. Particularly when dispensing cells or particles, the problem exists that these are not distributed homogeneously in the liquid, thus precluding the possibility to fill e.g. the wells of a titration plate with a reproducible number of cells or particles with small variance because, due to the occurrence of sedimentation effects and the like reasons, the concentration of the cells or particles in the suspensions will be locally altered. Thereby, the number of the dosed cells or particles is changed in each dosing process to such an extent that the measurement results are rendered unreliable. According to the invention, for homogenizing the particle distribution in the particle suspension, the dispensing device is provided with a suspension means acting on the particle suspension. The suspension means effects a mixing of the particle suspension so that a substantially homogeneous particle distribution in the particle suspension will be obtained. The variance of the number of the individual particles or cells in the wells will thus be extremely small. Preferably, the variance between the individual wells is smaller than 15%, and more preferably smaller than 10%. Such a small variance can also be realized during continuous dispensation over a period of several hours. For instance, the cell concentration in the suspension is between 1×10⁵ and 2×10⁶ per ml. This will result in a number of 100-2,000 cells per well in case of a filling quantity of 1 μl per well. Even with such high cell concentrations, the above mentioned low variance can be obtained through the provision of a suspension means in the reservoir.

Further, the dispensing device comprises a microdispenser for the dispensing of microdroplets. The microdroplets, being of a volume smaller than 5 μl, preferably smaller than 100 nl, more preferably smaller than 10 nl, still more preferably smaller than 3 nl and most preferably smaller than 0.5 nl, are dispensed by the microdispenser into the wells or recesses of a sample carrier. For this purpose, the microdispenser is connected to the reservoir via a fluid element for continuous supply of particle suspension to the microdispenser.

The inventive dispensing device makes it possible, due to the provision of a suspension means, to dispense a particle suspension, particularly a cell suspension, over a period of several hours with only low variance of the number of particles or cells in the individual dispensing processes. Thus, with the inventive dispensing device, one can generate a uniform and reproducible number of particles or cells in dependence from the supplied volume.

Particularly, the suspension means is of a suitable type to substantially preclude damage to the particles, especially the cells, during the mixing of the particle suspension.

To obtain the above effect, use can be made of a rod-type stirrer, for instance. This rod-type stirrer, which can be e.g. a rod-type stirrer with triangular stirring element, is driven e.g. by magnetic forces generated by a motor arranged below the bottom of the reservoir. In this manner, an extremely effective suspension can be obtained in the particle suspension. Since, however, in such a rod-type stirrer, friction will occur between the stirring element and the bottom of the reservoir, the resultant high shear forces may cause destruction of the cells. Thus, the suspension means is preferably provided as a suspended stirring system, particularly a suspended rod-type stirrer. This stirrer is installed in the reservoir, if possible in a centered position, via a rotatable axis connected e.g. to the cover of the reservoir. The driving motion can be imparted by a motor connected to the axis and arranged externally of the reservoir. The motor can also be arranged below the bottom of the reservoir and drive the rod-type stirrer by magnetic forces. To allow for the best possible driving of the stirrer by use of magnetic forces, the stirrer preferably comprises rare-earth metals. By the provision of the rare-earth metals, the driving force can be imparted to the stirrer also through thick reservoir walls, particularly through thick reservoir bottoms. A rod-type stirrer arranged in free suspended attachment in the particle suspension offers the advantage that the stirring can be performed without the stirrer touching the bottom or a wall of the reservoir. This will minimize the mechanical stresses acting on the cells or other particles included in the particle suspension while obtaining a good homogeneity within the particle suspension.

The stirrer of the preferably used type comprises at least two, preferably four stirring blades. These are arranged preferably at uniform distances on the circumference of the stirrer. The individual stirring blades are preferably arranged at an angle of 50 to 85°, and more preferably 60 to 70° relative to the longitudinal axis of the stirrer. This angle will be determined to the effect that the stirring blades are arranged at an angle of 40 to 5° and 30 to 20°, respectively, relative to a plane extending horizontally when the stirrer is arranged vertically. As compared to this horizontal plane, the inclination of the stirring blades is thus relatively flat. This has the advantage that the thrust between the particles in the particle suspension and the stirring blades is reduced. This thrust would be largest if the stirring blades were arranged in parallel to the longitudinal axis. Preferably, the stirring blades have rounded edges.

To further reduce the thrust between the particles and the stirring blades, the mixing faces of the mixing blades are of a slightly convex shape. The mixing faces are defined as those faces of the stirring blades onto which the particles impinge during the mixing.

The stirrer is preferably made from inert biocompatible materials or is coated with such materials. Preferably, the stirrer is provided with a PTFE coating. Further, the surface of the stirring blades is preferably as smooth as possible to avoid damage to the particles.

Particularly in case of dispensing cell particles over a period of several hours, the gas supply to the cells, particularly the supply with oxygen and carbon dioxide, is critical. Further, the supply of carbon dioxide serves for the pH regulation of the liquid. According to a preferred embodiment, the dispensing device of the invention comprises, in addition to the suspension means which does already enhance the gas infeed to the cells or particles, with a gas supply device. For this purpose, the reservoir is connected to the a gas supply device e.g. via a gas conduit. Preferably, the gas supply device is of a type which can be controlled to supply gas to the reservoir in dependence from e.g. the particle type. To this end, the reservoir is preferably configured such that it is not filled completely with the particle suspension and a gas space is formed in the upper region of the reservoir.

To further improve the supply of particles, especially the cells, with oxygen, carbon dioxide or other gases, the gas can also be introduced into the liquid so that the gas will form bubbles which will rise in the liquid, thus safeguarding the gas supply to the particles. However, it has been found that the introduction of gases into the liquid disadvantageously entails the risk of shear effects on the cells caused by the rising and possibly bursting gas bubbles. Preferably, in the inventive device, the gas to be supplied to the particles, especially the cells, is not introduced directly into the particle suspension but only into the reservoir so that the corresponding gases will be introduced into the space between the surface of the liquid and the cover. The movement of the particle suspension effected by the suspension means will bring about a sufficient intake of the required gases into the suspension.

To guarantee the dispensing of the particle suspension over a period of several hours, the reservoir preferably has a volume of 1-500 ml, with the size of the reservoir being adapted to the volumes of the wells provided e.g. in titration plates.

The microdispenser provided for the dispensation of the particle suspension is preferably activated piezoelectrically. This offers the advantage that very small microdroplets can be generated whose volume is smaller than 10 nl, preferably smaller than 3 nl and most preferably smaller than 0.5 nl. Further, the microdispenser can be configured to the effect that one piezoelement acts onto a flexible channel, which channel preferably is a round hose closed in itself, or the like. Thus, advantageously, the microdispenser can be cleaned in a simple manner and thereby kept in a sterile condition.

The microdispenser is provided e.g. as a glass-silicon structure having a pump chamber arranged therein. The pump chamber comprises an elastic chamber wall which is connected to an electrically controllable actuator provided to generate pressure in the pump chamber and to reduce the chamber volume, respectively. When the actuator is suitably controlled, the microdispenser will dispense a droplet. The pump chamber is preferably delimited by an inlet opening and an outlet opening which forms the outlet opening of the microdispenser or is connected thereto. The inlet opening is connected to the reservoir via a fluid element such as a hose. By the provision of an inlet opening, it is prevented that the pressure generated by the actuator in the pump chamber causes a displacement of liquid in the direction of the reservoir instead of the direction of the outlet opening. Via the inlet opening, a continuous inflow of the to-be-dispensed liquid is safeguarded. The pump chamber has a volume preferably between 100 nl and 5 ml, more preferably between 100 nl and 10 μl and most preferably between 500 nl and 2 μl. The nozzle width of the outlet opening is preferably smaller than 500 μm, more preferably smaller than 200 μm and most preferably smaller than 100 μm. Thus, with the preferred dispensing device, one can generate a drop size of a volume smaller than 10 nl, preferably smaller than 3 nl and most preferred smaller than 0.5 nl.

Further, the microdispenser can be a piston-type dispensing unit with valve and nozzle, or the like. By suitable control of the piston and the valve, it is likewise possible to generate very small droplets. In this arrangement, the piston space corresponding to the above pump chamber also has the above described dimensions so that droplet sizes corresponding to those mentioned above can be generated also here.

According to a preferred embodiment, the fluid element arranged between the reservoir and the microdispenser is provided with a shut-off unit. The shut-off unit serves for the interruption of the supply of the particle suspension to the microdispenser. Thus, it is possible to clean the microdispenser. Cleaning is performed e.g. at fixed time intervals. Preferably, the shut-off unit is a component which does not immediately interfere with the fluid system. This has the advantage that the sterility demands required for the handling of particle suspensions are fulfilled. The provision of a contactless shut-off unit further has the advantage that the shut-off unit is biocompatible. This means that no valve material, especially no metal particles, can come into contact e.g. with the cell suspension and affect the same. A particularly suited shut-off unit is a tube squeeze valve.

To facilitate the cleaning of the microdispenser, the microdispenser is preferably connected, via a second fluid element, to a supply container having a rinsing liquid provided therein. Thus, an automatic cleaning of the microdispenser can be performed. For this purpose, preferably, the second fluid element has a second shut-off unit connected thereto which can have the same configuration as the above described first shut-off unit. Also, a conventional valve can be provided in the second fluid unit or at the outlet of the supply container for the rinsing liquid because lower sterility demands have to be kept for the rinsing liquid.

Preferably, the inner diameters of the fluid elements and the shut-off devices do not or only slightly differ from each other. Thereby, a sedimentation of the particles and thus a de-homogenizing of the particle suspension are avoided. The diameters of the whole fluid system which preferably do not or only slightly differ from each other, are preferably in the range between 45 mm and 500 μm. Most preferred are diameters between 1.5 mm and 200 μm.

Preferably, the diameters of the individual channels provided in the device, particularly in dependence from the dispensed quantity of liquid, are selected to the effect that the dwelling time of the particle suspension, particularly the cell suspension, in the system is shorter than 10 min., preferably shorter than 5 min. and most preferably shorter than 2 min. Thus, sedimentation effects in the system are substantially avoided. These dwelling times are of course not valid for the reservoir in which the sedimentation is avoided by the inventive provision of a suspension agent.

A preferred embodiment of the invention will be explained in greater detail hereunder with reference to the accompanying drawing.

In the drawing:

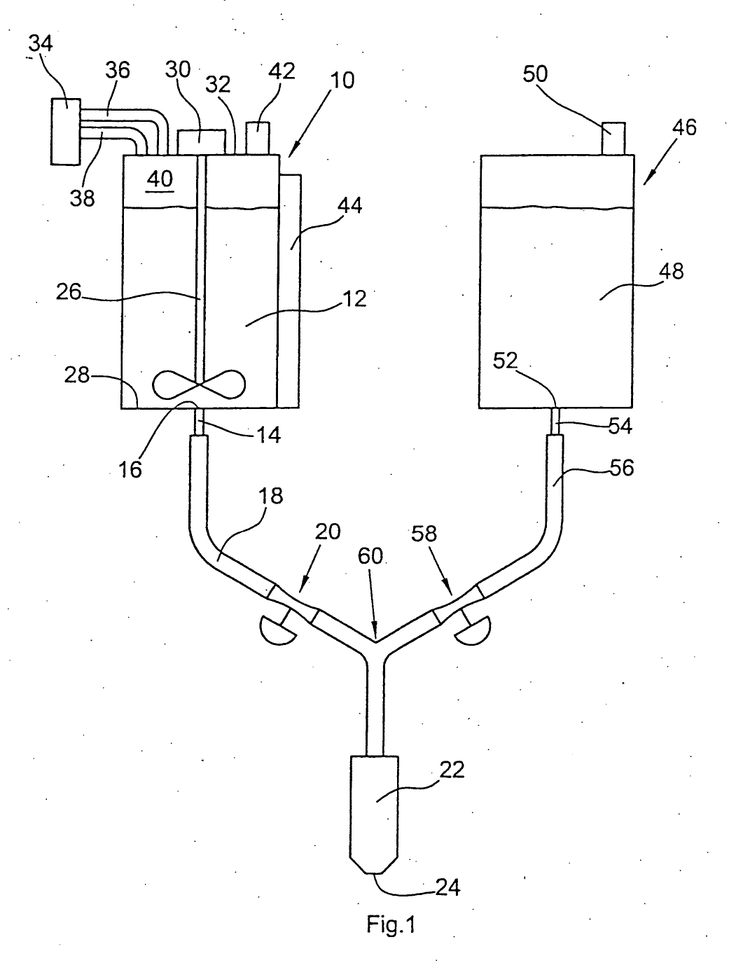

FIG. 1 is a schematic representation of an embodiment of the dispensing device according to the invention.

The dispensing device comprises a reservoir 10 in which the particle suspension 12 is received. The reservoir 10 has an outlet 16 provided with an outlet connector piece 14. The connector piece 14 has a hose 18 connected thereto as a fluid element. The hose is connected to a microdispenser 22 with a tube squeeze valve 20 interposed along the hose.

The inner diameters of hose 18 as well as of outlet connector piece 14 are preferably identical. Thus, the contact element is free of a dead volume. An identical connection exists also to the tube squeeze valve 20. By the provision of substantially constant inner diameters of the fluid elements, the occurrence of restriction regions which would form a flow resistance to the particles of the suspensions, is prevented. In case of such resistance, shear effects would occur, and the number of particles transported in the particle suspension would vary over time. This would lead to the so-called keystone effect, causing an non-reproducible number of cells or particles in the wells.

The microdispenser 22 has an outlet opening 24 provided to eject the microdroplets for filling the wells of a titration plate.

The suspension means of reservoir 10 is arranged as rod-type stirrer 26 by which the particle distribution in particle suspension 12 is kept homogenous throughout an extended period of preferably more than 6 hours, more preferably more than 10 hours.

Instead of the suspended rod-type stirrer 26 illustrated according to the instant embodiment, also a plurality of suspension means can be provided. Preferably, these are also located internally of reservoir 10 and therein are either supported on the reservoir bottom 28 or are mounted in a suspended arrangement within particle suspension 12. The driving of the suspension means can be performed, as in the case of the rod-type stirrer 26 in the illustrated embodiment, by means of a motor 30. Motor 30 is arranged externally of reservoir 10. In the illustrated embodiment, motor 30 is arranged externally on a cover 32 of reservoir 10. Further, it is possible to drive the suspension means in a contactless manner or through magnetic actuation, for instance.

A further possibility for the intermixing of the particle suspension and thus for the maintenance of a homogeneous particle distribution resides in generating a flow within the reservoir 10. This can be accomplished e.g. by the provision of circulating pumps. For this purpose, preferably two reservoirs are provided between which the particle suspension is moved by a circulating pump.

The reservoir 10 is connected to a gas supply device 34 for supplying gas to the particle suspension 12, particularly the cell suspension. By means of the gas supply device 34, gas, e.g. oxygen or carbon monoxide, is fed via conduits 36,38 into a gas chamber 40. The gas chamber 40 is arranged internally of reservoir 10 and is provided above the particle suspension 12. For control of the quantity of gas in the gas chamber 40 and of the different gas components, the gas supply device 34 is provided with a suitable control means. Further, a pressure control element 42 is arranged on the cover 32 for measuring the gas pressure in the gas chamber 40 and to control the pressure through the gas supply device 34.

Preferably, for tempering the particle suspension, a tempering element 44 is provided which in the illustrated embodiment is arranged on an outer wall of reservoir 10. The tempering element 44 serves for the heating and/or cooling of the particle suspension 12.

Further, the illustrated embodiment of the dispensing device of the invention comprises a supply container 46 containing a rinsing liquid 48. The pressure container 46 is also connected to a pressure control element 50 for pressure control. The pressure control element 50 makes it possible to generate an underpressure or overpressure in supply container 46.

An outlet 52 of supply container 46 is provided with an outlet connecting piece 54 which is connected to a tube 56. Tube 56 is connected, via a second tube squeeze valve 58, also with microdispenser 22. In the illustrated embodiment, the connection is effected via a Y-type tube connection member 60 so that the two tubes 18,56 are joined upstream of the microdispenser 22 and only one tube is connected to microdispenser 22.

1. A dispensing device for the dispensing of particle suspensions (12), particularly of cell suspensions, comprising
a reservoir (10) for receiving the particle suspension (12),
a suspension means (26) acting on the particle suspension (12) for maintaining a substantially homogeneous particle distribution in the particle suspension (12),
a microdispenser (22) for dispensing microdroplets of a volume smaller than 5 μl, and
a fluid element (18) connecting the reservoir (10) to the microdispenser (22) for continuous provision of particle suspension (12) at the microdispenser (22),
the suspension means being provided as a stirrer (26).

2. The dispensing device according to claim 1, characterized in that the suspension means (26) is configured to the effect that substantially no damage is caused to the particles, particularly the cells.

3. The dispensing device according to claim 1 or 2, characterized in that the stirrer comprises at least three, preferably at least four stirring blades.

4. The dispensing device according to claim 3, characterized in that the stirring blades include an angle of 50 to 85°, preferably 60 to 70° relative to a longitudinal axis of the stirrer (26).

5. The dispensing device according to claim 3 or 4, characterized in that the stirring blades comprise mixing faces of a slightly concave shape.

6. The dispensing device according to any one of claims 1-5, characterized in that the reservoir (10) is connected to a gas supply device for gas supply to the particle suspensions.

7. The dispensing device according to any one of claims 1-6, characterized in that the microdispenser (22) is piezoelectrically activated.

8. The dispensing device according to any one of claims 1-7, characterized in that the microdispenser comprises a pump chamber having a volume between 100 nl and 5 ml, more preferably between 100 nl and 10 μl and most preferably between 500 nl and 2 μl.

9. The dispensing device according to any one of claims 1-8, characterized in that the microdispenser comprises a dispensing opening (24) having a diameter smaller than 500 μm, preferably smaller than 200 μm and most preferably smaller than 100 μm.

10. The dispensing device according to any one of claims 1-9, characterized in that the fluid element (18) has a shut-off device (20) connected thereto for interrupting the supply of particle suspension (12) to the microdispenser (22).

11. The dispensing device according to claim 10, characterized in that the shut-off device is provided as a tube squeeze valve (20).

12. The dispensing device according to any one of claims 1-11, characterized in that the microdispenser (22) is connected, via a second fluid element (56), to a supply container (46) for rinsing liquid (48) for cleaning the microdispenser (22).

13. The dispensing device according to claim 12, characterized in that the second fluid element (56) is connected to a shut-off device (58).
